(19) United States
(12) Patent Application Publication
(10) Pub. No.: US 2007/0238725 A1
(43) Pub. Date: Oct. 11, 2007

(54) Therapeutic combinations for the treatment or prevention of psychotic disorders

(75) Inventor: Sharon Rosenzweig-Lipson, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/726,927

(22) Filed: Mar. 23, 2007

Related U.S. Application Data
(60) Provisional application No. 60/785,449, filed on Mar. 24, 2006.
(60) Provisional application No. 60/788,392, filed on Mar. 31, 2006.

(51) Int. Cl. A61K 31/551; A61K 31/498

(52) U.S. Cl. 514/220; 514/250

(57) ABSTRACT

Therapeutic combinations useful in the treatment or prevention of psychotic disorders, to pharmaceutical compositions containing said combinations, and to their use in the treatment or prophylaxis of psychotic disorders are provided. Such compounds are of formula I: 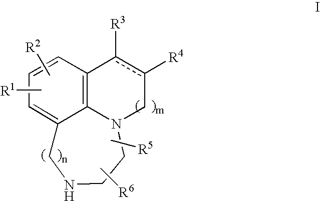
or a pharmaceutically acceptable salt thereof, wherein each of R¹, R², R³, R⁴, R⁵, R⁶, n, and m are as defined and described herein.

### CROSS-REFERENCE TO RELATED APPLICATIONS

The present application claims priority to U.S. provisional patent application Ser. No. 60/785,449, filed Mar. 24, 2006, and U.S. provisional patent application Ser. No. 60/788,392, filed Mar. 31, 2006, the entirety of each of which is hereby incorporated herein by reference.

### FIELD OF THE INVENTION

The present invention relates to therapeutic combinations of compounds useful for the treatment or prophylaxis of psychotic disorders, to pharmaceutical compositions containing such combinations, and to their use in the treatment or prophylaxis of psychotic disorders.

### BACKGROUND OF THE INVENTION

Psychoses are serious mental illnesses characterized by defective or lost contact with reality. The symptoms associated with these disorders are classified as positive symptoms (disordered thought, hallucinations, and delusions), negative symptoms (social withdrawal and unresponsiveness), and cognitive deficits.

A variety of drugs are available for the treatment of psychotic disorders. For example, neuroleptics or anti-psychotics can be used to treat schizophrenia and other psychotic disorders by blocking the dopaminergic neurotransmission in the central nervous system.

Neuroleptics are used widely to treat the “positive” symptoms of schizophrenia. However, many of these drugs are not considered to be effective for the treatment of “negative” symptoms of schizophrenia and may in fact exacerbate these symptoms because of the dopaminergic blockade associated with their mechanism of action. Cognitive deficits associated with schizophrenia, such as distractability, and executive skills such as a working memory and ability to plan, are also believed to be negatively effected by the blockade of dopamine receptors. In addition, these neuroleptics have important side effects such as akathisia, dystonia, Parkinsonism dyskinesia and late dyskinesia and the like, which are caused by blocking dopaminergic neurotransmission.

Anticholinergic agents such as Cogentin®, have been used to reduce Parkinson-like side effects, but also cause side effects such as mental and/or physical impairment, tachycardia, dysuria and gastrointestinal symptoms.

Despite the variety of treatment options available, there remain significant difficulties successfully treating the different symptoms of psychotic disorders while minimizing side effects; there remains a need for the development of new therapeutic strategies.

### SUMMARY OF THE INVENTION

The present invention provides new combination therapies for the treatment of psychotic disorders. In particular, the present invention demonstrates that combinations of a 5HT_{2C} agonist, or partial agonist, with one or more anti-psychotic agents are useful for treating patients suffering from or susceptible to one or more psychotic disorders. The present invention therefore provides, among other things, certain drug combinations, pharmaceutical compositions containing such combinations, and methods of treating patients suffering from or susceptible to one or more psychotic disorders with such combinations or compositions.

### BRIEF DESCRIPTION OF THE DRAWING

FIG. 1 shows the effects of Compound 1, alone or in combination with haloperidol, on apomorphine-induced climbing.

FIG. 2 shows the effects of Compound 1, alone or in combination with clozapine, on apomorphine-induced climbing.

FIG. 3 shows the effects of Compound 2, alone or in combination with haloperidol, on apomorphine-induced climbing.

FIG. 4 shows the effects of Compound 2, alone or in combination with clozapine, on apomorphine-induced climbing.

FIG. 5 shows the effect of Compound 1 on the potentiation of haloperidol's effect on conditioned aviodance responding.

FIG. 6 shows the effect of Compound 1 on the potentiation of clozapine's effect on conditioned aviodance responding.

FIG. 7 shows the effect of Compound 2 on the potentiation of haloperidol's effect on conditioned aviodance responding.

FIG. 8 shows the effect of Compound 2 on the potentiation of clozapine's effect on conditioned aviodance responding.

### DETAILED DESCRIPTION OF CERTAIN EMBODIMENTS OF THE INVENTION

The present invention encompasses the finding that 5-HT_{2C} receptor agonists, or partial agonists, can be usefully combined with other anti-psychotic agents for the treatment or prevention of anti-psychotic disorders. In particular, the present invention provides the surprising finding that combinations of 5-HT_{2C} receptor agonists, or partial agonists, with either typical or atypical anti-psychotic drugs shows increased efficacy, without increased side effects, in the treatment of anti-psychotic disorders. Accordingly, on aspect of the present invention provides a composition comprising a 5-HT_{2C} receptor agonist, or partial agonist, and either a typical or atypical anti-psychotic drug.

In certain embodiments, the present invention provides the surprising finding that combinations of 5-HT_{2C} receptor agonists, or partial agonists, of formula I: 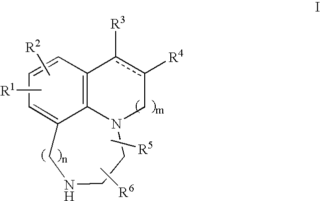
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
   each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R;
   with either typical or atypical anti-psychotic drugs shows increased efficacy, without increased side effects, in the treatment of anti-psychotic disorders.

5-HT_{2C} receptor agonists of formula I inhibit levels of mesolimbic dopamine and can act as effective anti-psychotic agents in their own right. Typical anti-psychotic drugs (e.g., haloperidol) block both mesolimbic and nigrostriatal dopamine and thus treat the positive symptoms of psychotic disorders, which are associated with mesolimbic dopamine, but also produce extra-pyramidal side effects resulting from inhibition of nigrostriatal dopamine. Atypical anti-psychotic drugs (e.g., clozapine) selectively block mesolimbic dopamine. Such agents are less prone to extra-pyramidal side effects, but have a myriad of other side effect liabilities including weight gain.

The present invention demonstrates that co-administration of sub-effective doses of 5-HT_{2C} receptor agonists of formula I and sub-effective doses of typical anti-psychotic drugs results in increased efficacy with no increase in extra-pyramidal side effects. Furthermore, the present invention demonstrates that co-administration of sub-effective doses of atypical anti-psychotic agents also results in increased efficacy. Agonism of the 5-HT_{2C} receptor may also alleviate the weight gain and/or other side effects associated with such agents. Thus, the present invention provides improved combination therapies for the treatment of anti-psychotic disorders. The improved efficacy of the inventive combinations, particularly as associated with reduced side effects, should have the further benefit of improved patient compliance with the treatment regimen.
1. 5-HT_{2C} Receptor Agonists of Formula I

The present invention utilizes 5-HT_{2C} receptor agonists, or partial agonists, of formula I: 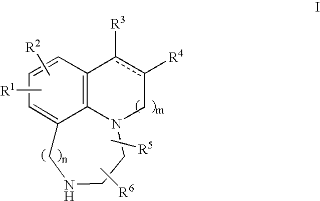
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
   each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R;
   in combination with one or more anti-psychotic agents.

As used herein, the term “alkyl” includes, but is not limited to, straight and branched chains such as methyl, ethyl, n-propyl, isopropyl, n-butyl, isobutyl, sec-butyl, or t-butyl.

The terms “halogen” or “halo,” as used herein, refer to chlorine, bromine, fluorine or iodine.

The term “perfluoroalkyl,” as used herein refers to an alkyl group, as defined herein, wherein every hydrogen atom on said alkyl group is replaced by a fluorine atom. Such perfluoroalkyl groups include —CF₃.

The terms “effective amount” and “therapeutically effective amount,” as used herein, refer to the amount of a compound or combination that, when administered to an individual, is effective to treat, prevent, delay, or reduce the severity of a condition from which the patient is suffering. In particular, a therapeutically effective amount in accordance with the present invention is an amount sufficient to treat, prevent, delay onset of, or otherwise ameliorate at least one symptom of a psychotic disorder or episode.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

The term “patient,” as used herein, refers to a mammal. In certain embodiments, the term “patient” refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The compounds of formula I, as defined above or in classes and subclasses as described herein, have affinity for and agonist or partial agonist activity at the 2C subtype of brain serotonin receptors.
2. Description of Exemplary Compounds:

In certain embodiments, [symbol] designates a single bond. In other embodiments, [symbol] designates a double bond.

In certain embodiments, the R¹ group of formula I is R, OR, halogen, cyano, or —C₁₋₃ perfluoroalkyl. In other embodiments, the R¹ group of formula I is hydrogen, halogen, cyano, —OR wherein R is C₁₋₃ alkyl, or trifluoromethyl. According to another embodiment, the R¹ group of formula I is hydrogen.

In certain embodiments, the R² group of formula I is R, OR, halogen, cyano, or —C₁₋₃ perfluoroalkyl. In other embodiments, the R² group of formula I is hydrogen, halogen, cyano, —OR wherein R is hydrogen, C₁₋₃ alkyl, or trifluoromethyl. According to another embodiment, the R² group of formula I is hydrogen.

According to one aspect of the present invention, at least one of R¹ and R² groups of formula I is —OH. According to another aspect of the present invention, both of the R¹ and R² groups of formula I are —OH.

According to another embodiment, each of the R¹ and R² groups of formula I is hydrogen. According to yet another embodiment, each of the R⁵ and R⁶ groups of formula I is hydrogen.

As defined generally above, the R³ and R⁴ groups of formula I are taken together to form a saturated or unsaturated 4-8 membered ring, wherein said ring is optionally substituted with 1-3 groups independently selected from halogen, —R, or OR. According to one embodiment, the R³ and R⁴ groups of formula I are taken together to form a saturated or unsaturated 5-8 membered ring, wherein said ring is optionally substituted with 1-3 groups independently selected from halogen, —R, or OR. In certain embodiments, the R³ and R⁴ groups of formula I are taken together to form a saturated or unsaturated 5-6 membered ring, wherein said ring is optionally substituted with 1-3 groups independently selected from halogen, —R, or OR.

As defined generally above, n is 1 or 2. Accordingly, the present invention provides a compound of formulae I-a and I-b: 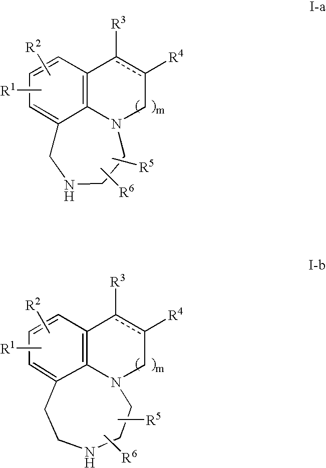
or a pharmaceutically acceptable salt thereof, wherein each of m, R¹, R², R³, R⁴, R⁵, and R⁶ is as defined above for compounds of formula I and described in classes and subclasses above and herein.

As defined generally above, m is 0 or 1. Accordingly, the present invention provides a compound of formulae I-c and I-d: 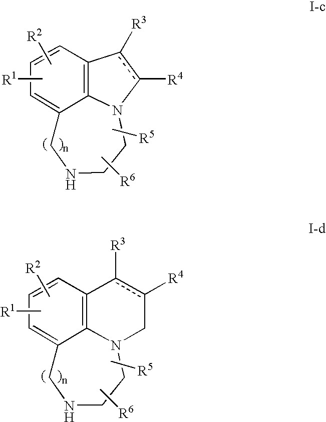
or a pharmaceutically acceptable salt thereof, wherein each of n, R¹, R², R³, R⁴, R⁵, and R⁶ is as defined above for compounds of formula I and described in classes and subclasses above and herein.

In other embodiments, n is 1, m is 1, and the R³ and R⁴ groups of formula I are taken together to form a saturated 5-membered ring and said compound is of formula II: 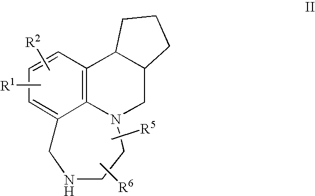
or a pharmaceutically acceptable salt thereof, wherein each of R¹, R², R⁵, and R⁶ is as defined above for compounds of formula I and described in classes and subclasses above and herein.

According to another aspect of the present invention, a compound is provided, wherein n is 1, m is 0, and the R³ and R⁴ groups of formula I are taken together to form a saturated 5-membered ring and said compound is of formula III: 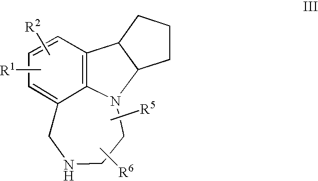
or a pharmaceutically acceptable salt thereof, wherein each of R¹, R², R⁵, and R⁶ is as defined above for compounds of formula I and described in classes and subclasses above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

According to another aspect, the present invention provides a compound of either of formulae I-e or I-f: 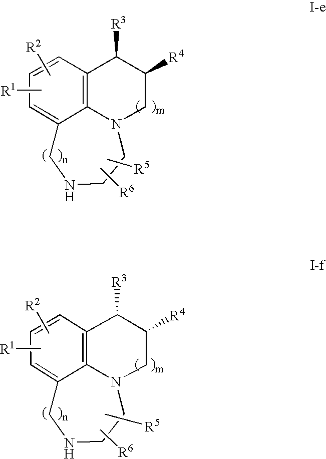
or a pharmaceutically acceptable salt thereof, wherein each of n, m, R¹, R², R³, R⁴, R⁵, and R⁶ is as defined above for compounds of formula I and described in classes and subclasses above and herein.

In certain embodiments, the present invention provides a compound of either of formulae IV or V: 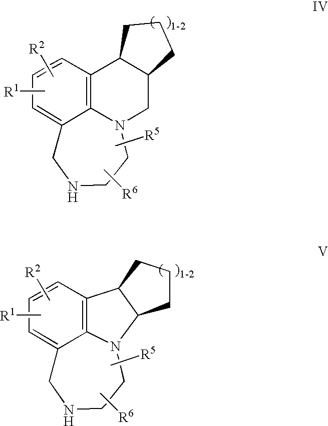
or a pharmaceutically acceptable salt thereof, wherein each R¹, R², R⁵, and R⁶ are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Exemplary compounds useful for the methods of the present invention are set forth in Table 1, below.

TABLE 1
Exemplary Compounds of Formula I

2-bromo-4,5,6,7,9,9a,10,11,12,12a-decahydrocyclopenta[c][1,4]diazepino [6,7,1-ij]quinoline;
2-bromo-4,5,6,7,9,9a,10,11,12,13,14,14a-dodecahydrocyclohepta[c][1,4]diazepino[6,7,1-
ij]quinoline;
2-chloro-4,5,6,7,9,9a,10,11,12,12a-decahydrocyclopenta[c][1,4]diazepino [6,7,1-ij]quinoline;
2-chloro-4,5,6,7,9,9a,10,11,12,13,14,14a-dodecahydrocyclohepta[c][1,4]diazepino[6,7,1-
ij]quinoline;
2-phenyl-4,5,6,7,9,9a,10,11,12,12a-decahydrocyclopenta[c][1,4]diazepino [6,7,1-ij]quinoline;
2-methoxy-4,5,6,7,9,9a,10,11,12,12a-decahydrocyclopenta[c][1,4]diazepino [6,7,1-ij]quinoline;
1-fluoro-4,5,6,7,9,9a,10,11,12,12a-decahydrocyclopenta[c][1,4]diazepino [6,7,1-ij]quinoline;
1-fluoro-4,5,6,7,9,9a,10,11,12,13,14,14a-dodecahydrocyclohepta[c][1,4] diazepino[6,7,1-
ij]quinoline;
1-(trifluoromethyl)-4,5,6,7,9,9a,10,11,12,12a-decahydrocyclopenta[c][1,4] diazepino[6,7,1-
ij]quinoline;
1-fluoro-2-methoxy-4,5,6,7,9,9a,10,11,12,12a-decahydrocyclopenta[c][1,4] diazepino[6,7,1-
ij]quinoline;
1-fluoro-2-methoxy-4,5,6,7,9,9a,10,11,12,13,14,14a-dodecahydrocyclohepta
[c][1,4]diazepino[6,7,1-ij]quinoline;
4,5,6,7,9,9a10,11,12,12a-decahydrocyclopenta[c][1,4]diazepino[6,7,1-ij] quinoline;
4,5,6,7,9,9a,10,11,12,13,14,14a-dodecahydrocyclohepta[c][1,4]diazepino [6,7,1-ij]quinoline;
(—)-4,5,6,7,9,9a,10,11,12,12a-decahydrocyclopenta[c][1,4]diazepino[6,7,1-ij] quinoline;
(9aR,14aS)-4,5,6,7,9,9a,10,11,12,13,14,14a-dodecahydrocyclohepta[c][1,4] diazepino[6,7,1-
ij]quinoline;
(9aS,14aR)-4,5,6,7,9,9a,10,11,12,13,14,14a-dodecahydrocyclohepta[c][1,4] diazepino[6,7,1-
ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7,1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
2S)-(rel-7bR,10aR)-2-methyl-1,2,3,4,8,9,10,10a-octahydro-7bH-cyclopenta[b]
[1,4]diazepino[6,7,1-hi]indole;
(2S)-(rel-7bR,10aR)-2-methyl-1,2,3,4,8,9,10,10a-octahydro-7bH-cyclopenta[b]
[1,4]diazepino[6,7,1-hi]indole;
(2S)-(rel-7bS,10aS)-2-methyl-1,2,3,4,8,9,10,10a-octahydro-7bH-cyclopenta[b]
[1,4]diazepino[6,7,1-hi]indole;
(2R)-(rel-7bR,10aR)-2-methyl-1,2,3,4,8,9,10,10a-octahydro-7bH-cyclopenta[b]
[1,4]diazepino[6,7,1-hi]indole;
(2R)-(rel-7bR,10aR)-2-methyl-1,2,3,4,8,9,10,10a-octahydro-7bH-cyclopenta[b]
[1,4]diazepino[6,7,1-hi]indole;
(2R)-(rel-7bS,10aS)-2-methyl-1,2,3,4,8,9,10,10a-octahydro-7bH-cyclopenta[b]
[1,4]diazepino[6,7,1-hi]indole;
rel-(4S,7bS,10aS)-4-methyl-1,2,3,4,8,9,10,10a-octahydro-7bH-
cyclopenta[b][1,4]diazepino[6,7,1-hi]indole
rel-(4S,7bS,10aS)-4-methyl-1,2,3,4,8,9,10,10a-octahydro-7bH-cyclopenta[b]-
[1,4]diazepino[6,7,1-hi]indole;
rel-(4R,7bS,10aS)-4-methyl-1,2,3,4,8,9,10,10a-octahydro-7bH-
cyclopenta[b][1,4]diazepino[6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]diazepino[6,7,1-
hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4] diazepino[6,7,1-
hi]indole; and
(7bS,10aS)-9,9-dimethyl-1,2,3,4,8,9,10,10a-octahydro-7bH-cyclopenta[b][1,4] diazepino[6,7,1-
hi]indole;

or a pharmaceutically acceptable salt thereof. Another aspect of the present invention provides the hydrochloride salt of each of the above compounds.

Also, it will be appreciated by those of ordinary skill in the art that reference to a compound herein is intended to include reference to any and all related forms such as polymorphs, hydrates, etc. Also, compounds may be provided as pro-drugs or other forms converted into the active agent during manufacture, processing, formulation, delivery, or in the body.

It will additionally be appreciated that the principles of the present invention apply all radiolabelled forms of the compounds receited herein, including, for example, those where the radiolabels are selected from as ³H, ¹¹C, ¹⁴C, ¹⁸F, ¹²³I and ¹²⁵I. Such radiolabelled compounds are useful as research and diagnostic tools in metabolism pharmacokinetics studies and in binding assays in both animals and humans.

Compounds of formula I for use in accordance with the present invention may be obtained or produced according to any available means including methods described in detail in WO 03/091250, published Nov. 6, 2003, and in WO 06/052768, published May 18, 2006, the entirety of each of which is hereby incorporated herein by reference.
3. Anti-Psychotic Agents

Anti-psychotic agents that may usefully be employed in inventive combinations include those that work as a full antagonist of the dopamine D2 receptor and include both typical and atypical anti-psychotics, or pharmaceutically acceptable salts of such agents. It will be understood that reference to “anti-psychotic agents”, “neuroleptic agents”, or to specific compounds having anti-psychotic activity, can include their pharmaceutically acceptable salts. Representative anti-psychotic agents that are commercially available or known to those skilled in the art and include, but are not limited to the following compound and their pharmaceutically acceptable salts:

TABLE 2
Exemplary Anti-psychotic Agents

| COMMON NAME | CHEMICAL NAME | REFERENCE* | EXEMPLARY DOSE |
|---|---|---|---|
| amisulpiride | 4-amino-N-[(1-ethyl-2- | U.S. Pat. No. | about 50-about 800 mg/ |
| | pyrrolidin-yl)methyl]-5- | 4,401,822 | day |
| | (ethyl-sulfonyl)-2- | | |
| | methoxybezamide | | |
| aripiprazole | Dihydroquinolinone | U.S. Pat. No. | about 2.5-about 30 mg/ |
| | | 4,734,416 | day |
| | | 5,00,528 | |
| chlorpromazine | 2-chloro-N,N-dimethyl- | U.S. Pat. No. | about 300-about |
| | 10H-phenothiazine-10- | 2,645,640 | 800 mg per day |
| | propanamine | | |
| clozapine | 8-chloro-11-(4-methyl-1- | U.S. Pat. No. | about 300-about |
| | piperazinyl)-5H- | 3,539,573 | 600 mg/day |
| | dibenzo[b,e]-[1,4]- | | |
| | diazepine | | |
| fluphenazine | 4-[3-[2-(trifluoromethyl)- | GB 829,246 | about 2-about 5 mg |
| | 10H-phenothiazin-10- | | per/day |
| | yl]propyl]-1- | | |
| | piperazineethanol | | |
| haloperidol | 4-[4-(4-chlorophenyl)-4- | U.S. Pat. No. | about 1-about 20 mg |
| | hydroxy-1-piperidinyl]-1- | 3,438,991 | per day |
| | (4-fluoro-phenyl)-1- | | |
| | butanone | | |
| loxapine | 2-chloro-11-(4-methyl-1- | U.S. Pat. No. | about 60-about 100 mg/ |
| | piperazinyl)- | 3,546,226 | day |
| | dibenz[b,f][1,4]oxazepine | | |
| mesoridazine | 10-[2-(1-methyl-2- | U.S. Pat. No. | about 100-about |
| | piperidinyl)ethyl]-2- | 3,084,161 | 400 mg/day |
| | (methylsulfinyl)-10H- | | |
| | pheno-thiazine | | |
| molindone | 3-ethyl-1,5,6,7- | U.S. Pat. No. | about 15-about 225 mg/ |
| | tetrahydro-2-methyl-5-(4-- | 3,491,093 | day |
| | morpholinylmethyl)-4H- | | |
| | indol-4-one | | |
| olanzapine | 2-methyl-4-(4-methyl-1- | U.S. Pat. No. | about 10-about 20 mg/ |
| | piperazinyl)-10H- | 5,229,382 | day. |
| | thieno[2,3-b][1,5]- | | |
| | benzodiazepine | | |
| perphenazine | 4-[3-(2-chloro-10H- | U.S. Pat. No. | about 8-about 40 mg/ |
| | phenothiazin-1- | 2,766,235 | day |
| | yl)propyl]-1-piperazine- | | |
| | ethanol | | |
| pimozide | 1-(1-(4,4-bis(4- | | about 2-12 mg/day |
| | fluorophenyl)butyl)-4- | | |
| | piperidinyl)-1,3-dihydro- | | |
| | 2H-benzimidazole-2-one | | |
| quetiapine | 11-[4-/2-(2- | U.S. Pat. No. | about 300-about |
| | hydroxyethoxy)ethyl/-1- | 4,879,288 | 600 mg/day |
| | piperazinyl]dibenzo[b,f]- | | |
| | 1,4-thiazepine | | |
| risperidone | 3-[2-[4-(6-fluoro-1,2- | U.S. Pat. No. | about risperidone |
| | benzisoxazol-3- | 4,804,663 | from about 4-about |
| | yl)piperidino]-ethyl]- | | 20 mg/day day |
| | imidazolidin-2-one | | |
| seroquel | 11-[4-[2-(2- | EP 240228 | about 15-about 750 mg/ |
| | hydroxyethoxy)ethyl]-1- | | day |
| | piperazinyl]dibenzo[b,f]- | | |
| | [1,4]thiazepine | | |
| sulpiride | 5-(aminosulfonyl)-N-[(1- | U.S. Pat. No. | about 400-800 mg/ |
| | ethyl-2- | 3,342,826 | day |
| | pyrolidinyl)methyl]-2- | | |
| | methoxybenzamide | | |
| thioridazine | 10-[2-(1-methyl-2- | Collect. Czech. | about 200-about |
| | piperidinyl)ethyl]-2- | Chem. Commun., | 600 mg/day |
| | (methylthio)-10H- | 55: 1586-1601, 1990 | |
| | phenothiazine | | |
| thiothixene | N,N-dimethyl-9-[3-(4- | U.S. Pat. No. | about 20-about 30 mg/ |
| | methyl-1-piperazinyl)- | 3,310,553 | day |
| | propylidene-9H- | | |
| | thioxanthene-2- | | |
| | sulfanamide | | |
| trifluoperazine | 10-[3-(4-methyl-1- | GB 813,861 | about 6-about 20 mg/ |
| | piperazinyl)-propyl]-2- | | day; |
| | (trifluoro-methyl)-10H- | | |
| | phenothiazine | | |
| ziprasidone | 5-(2-(4-(1,2- | U.S. Pat. Nos. | about 80-about 160 mg/ |
| | Benzisothiazol-3- | 4,831,031 | day |
| | yl)piperazinyl)ethyl)-6- | 5,312,925 | |
| | chloro-1,3-dihydro- | 6,150,366 | |
| | 2(1H)-indole-2-one | 6,245,766 | |

*Each cited reference is incorporated herein by reference in its entirety.

Other reported anti-psychotic agents that can usefully be employed in combination with compounds of formula I include, for example (S)-2-(benzylamino-methyl)-2,3,8,9-tetrahydro-7H-1,4-dioxino[2,3-e]indol-8-one, a D2 partial agonist, that is disclosed in U.S. Pat. No. 5,756,532; or pharmaceutically acceptable salts thereof. According to another embodiment, a compound of formula I is employed in combination with bifeprunox.

Anti-psychotic agents for use in accordance with the present invention may be obtained or produced according to any available means.
4. Pharmaceutical Compositions

While it is possible for the active ingredients of the inventive combination to be administered as the raw chemical, it is often desirable to present them in the context of one or more pharmaceutical formulations. Pharmaceutical formulations according to the present invention comprise a combination according to the invention together with one or more pharmaceutically acceptable carriers or excipients and optionally other therapeutic agents.

Thus, present invention also provides a pharmaceutical composition comprising one or more 5-HT_{2C} receptor agonists of formula I: 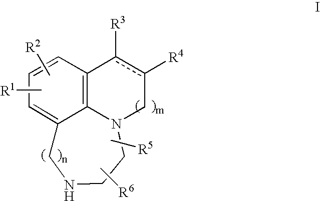
or a pharmaceutically acceptable salt thereof, wherein:
[symbol] designates a single or double bond;
n is 1 or 2;
m is 0 or 1;
R¹ and R² are each independently halogen, —CN, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R;
   and one or more anti-psychotic agents as a combined preparation for simultaneous, separate or sequential administration to treat a patient suffering from or susceptible to a psychotic disorder or episode.

Agents used in inventive combinations may be administered simultaneously, in the same or different pharmaceutical formulation, or sequentially. Of course the timing of the sequential administration should preserve the advantageous effects of the combination and said timing can be determined by a skilled practitioner.

A therapeutically effective amount of the combination will be understood to be an amount which treats, inhibits, prevents or ameliorates one or more symptoms of the psychotic disorder or episode in question. In certain embodiments of the invention, the combination will show improved efficacy than achieved by administration of the same amount of either the compound of formula I or the anti-psychotic agent alone. Furthermore, in certain embodiments the effective amount of the combination produces fewer side effects than are observed when the anti-psychotic agent is administered alone at a dose that achieves substantially similar therapeutic efficacy.

The dosages of each of the drugs in the combination may be determined by a physician and will often depend upon the specific psychotic disorder or episode, as well as the size, age and response pattern of the patient. Dosage guidelines are provided here. For the combination, the dosage guideline for each of the drugs of the combination would be considered.

In general, suitable doses of compound of formula I from about 0.5 mg per day to about 500 mg per day; in some embodiments from about 1 to about 500 mg per day.

A suitable dose of anti-psychotic agent may be in the range recommended by the manufacturer. In some embodiments of the invention, the anti-psychotic agent is used at the low end of the range recommended by the manufacturer, or even below the range, in light of synergistic benefits that can be achieved according to the present invention. Exemplary dosages for some preferred anti-psychotics are provided as guidelines in Table 2.

Useful carriers for use in inventive pharmaceutical formulations are compatible with the other ingredients in the composition. According to the present invention, compounds of formula I may be administered with anti-psychotic agents in a single pharmaceutical formulation, or in multiple formulations. Where multiple formulations are employed, each may include both the compound of formula I and the anti-psychotic agent, or alternatively, each may include only one.

An inventive combination of one or more compounds of formula I and one or more anti-psychotic agents may conveniently be presented as a pharmaceutical formulation in a unitary dosage form. A convenient unitary dosage formulation contains the active ingredients in amounts from 0.1 mg to 1 g each, for example 5 mg to 500 mg. Typical unit doses may, for example, contain about 0.5 to about 500 mg, or about 1 mg to about 500 mg, of a compound of formula I.

According to the present invention, pharmaceutical formulations may be prepared as “patient packs” containing the whole course of treatment in a single package, for example a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance

It will be understood that the administration of the inventive combination by means of a single patient pack, or patient packs of each formulation, with a package insert directing the patient to the correct use of the invention is a desirable additional feature of this invention.

According to a further aspect of the invention, there is provided a patient pack comprising at least one active ingredient of the combination of the invention and an information insert containing directions on the use of the combination of the invention.

According to the present invention, combinations of one or more compounds of formula I and one or more anti-psychotic agents may be formulated for any mode of delivery including, for example, oral, rectal, nasal, topical (including transdermal, buccal and sublingual), vaginal or parenteral (including subcutaneous, intramuscular, intravenous and intradermal) administration. The formulations may be prepared by any methods well known in the art of pharmacy, for example, using methods such as those described in Gennaro et al., Remington's Pharmaceutical Sciences (18th ed., Mack Publishing Company, 1990, see especially Part 8: Pharmaceutical Preparations and their Manufacture). Such methods typically include a step of bringing into association the active ingredient(s) with the carrier which constitutes one or more accessory ingredients. Such accessory ingredients include, for example, fillers, binders, diluents, disintegrants, lubricants, colorants, flavouring agents and wetting agents.

Formulations suitable for oral administration may be presented, for example, as discrete units such as pills, tablets or capsules each containing a predetermined amount of active ingredient; as a powder or granules; as a solution or suspension. The active ingredient may also be present as a bolus or paste, or may be contained within liposomes. Formulations suitable for oral administration may alternatively be presented, for example, as liquids. Liquid formulations may be particularly useful for administration to children. In general, when preparing liquid formulations for administration to children, it is desirable to avoid or minimize use of alcohol in the formulation.

Formulations for rectal administration may be presented, for example, as a suppository or enema.

For parenteral administration, suitable formulations include aqueous and non-aqueous sterile injection. The formulations may be presented in unit-dose or multi-dose containers, for example, sealed vials and ampoules, and may be stored in a freeze dried (lyophilized) condition requiring only the addition of the sterile liquid carrier, for example, water prior to use.

Formulations suitable for administration by nasal inhalation include, for example, fine dusts or mists which may be generated by means such as metered dose pressurized aerosols, nebulisers or insufflators.

Those of ordinary skill in the art will appreciate that pharmaceutical compositions comprising inventive combinations may further include one or more additional pharmaceutically active agents. For example, according to the present invention, the inventive combinations may be administered in conjunction with one or more other agents that is/are useful in treating psychotic discorders or their symptoms. Alternatively or additionally, inventive combinations may be administered with one or more other pharmaceutical agents active in treating any other symptom or medical condition experienced by the individual of interest, whether related or unrelated to the psychotic disorder from which the individual suffers. Examples of such pharmaceutical agents include, for example, pain relieving agents, anti-depressants, anti-anxiety drugs, and/or other agents that treat one or more mood disorders. Additional examples of such pharmaceutical agents include, for exqmple, anti-angiogenic agents, anti-diabetic agents, anti-infective agents, pain-relieving agents, gastrointestical agents, etc., or combinations thereof. A more complete list of such pharmaceutically active agents can be found in the Physicains' Desk Reference, 55^{th} Edition, 2001, published by Medical Economics Co., Inc., Montvale, N.J.
5. Uses

Administration of the inventive combinations is useful for the treatment or prevention of psychotic disorders or episodes. For example, according to the present invention, combinations of one or more compounds of formula I and one or more anti-psychotic agents may be used in the treatment of schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; psychosis associated with Lewy body disease; bipolar disorders such as bipolar I disorder, bipolar II disorder, and cyclothymic disorder; dementia, and depression with psychotic features. In some embodiments, inventive combinations are useful in the treatment of bipolar disorder, including for example treating the cycling between bipolar depression and bipolar mania. A more complete description of the aforementioned mental disorders can be found in the Diagnostic and Statistical Manual of Mental Disorders, 4th edition, Washington, D.C., American Psychiatric Association (1994), incorporated herein by reference in its entirety.

Alternatively or additionally, the inventive combinations may be employed to treat psythotic disorders, as described herein, with more rapid onset of benefit, and/or with fewer side effects. In certain embodiments, the present combinations are useful for treating psythotic disorders, as described herein, with a decreased level of sexual dysfunction. In other embodiments, the present combinations are useful for treating psythotic disorders, as described herein, and preventing the onset of sexual dysfunction. It was surprisingly found that compounds of the present invention provide a rapid onset of action as compared with other therapeutic agents typically used for treating schizoprenia and other psychotic disorders.

In particular, inventive combinations are useful in the treatment of psychotic disorders associated with altered neurotransmission activity of the dopaminergic system in the central nervous system. In some embodiments, the inventive combinations provide anti-psychotic benefits while eliminating or minimizing certain side affects (e.g., akathisia, dystonia, Parkinsonism dyskinesia and late dyskinesia and the like) associated observed when the anti- psychotic agent(s) is/are taken alone.

Combinations of the present invention are also useful for treating symptoms related to psychotic disorders of the schizophrenic types, including the so called “positive” and “negative” symptoms of schizophrenia. These symptoms include for example hallucinations, delusions, paranoia, anxiety, agitation, excessive aggression, tension, thought disorder, blunted affect, and social or emotional withdrawal in psychotic patients. Other symptoms often associated with psychotic disorders include cognition disorders or deficits such as poor attention and impaired function, depression, suicide, metabolic syndrome, and substance abuse. Thus, another embodiment of the present invention provides a method for treating one or more symptoms associated with a psychotic disorder.

In other embodiments, the present combinations are useful for treating anxiety disorders such as panic attack, agoraphobia, panic disorder, specific phobia, social phobia, social anxiety disorder, obsessive compulsive disorder, posttraumatic stress disorder, acute stress disorder, generalized anxiety disorder, separation anxiety disorder, substance-induced anxiety disorder, and anxiety disorder not otherwise specified.

According to another embodiment, the present combinations are useful for treating bipolar disorders. Such bipolar disorders include bipolar I disorder, bipolar II disorder, and cyclothymic disorder; bipolar mania, dementia, and depression with psychotic features. The present compounds are also useful for treating (including the preventing) of cycling that may occur between bipolar depression and bipolar mania.

Inventive combinations may be administered to patients suffering from or susceptible to one or more psychotic disorders or episodes, according to a treatment regimen and dosing plan established by a doctor. In general, a patient is considered to be suffering from a psychotic disorder if that patient shows an appropriate collection of accepted symptoms of that disorder. A patient is considered to be susceptible to a psychotic disorder or episode if, for example, that patient has a familial history of the disorder, or carries a known genetic susceptibility trait for that disorder. A patient may also be considered to be susceptible if the patient has shown one or more symptoms of the disorder, or has experienced an episode of the disorder, in the past.

In general, the term “treatment,” as used herein, refers to reversing, alleviating, delaying the onset of, inhibiting the progress of, or preventing a psychotic disorder or episode. In some embodiments, treatment may be applied after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Those of ordinary skill in the art will also recognize that inventive combinations and cpmpositions useful in the treatment of psychotic disorders may also find utility in the treatment of other disorders, for example depression or other mood disorders, many of which show significant co-morbidity with psychotic disorders.

### EXEMPLIFICATION

### 1. Example 1

### Inventive Combinations Reduce Apomorphine-Induced Climbing without Side Effects

Using 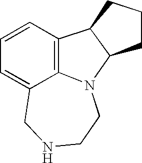
(Compound 1, “cpd 1”) and

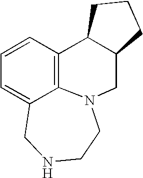

(Compound 2, “cpd 2”) to exemplify, the present Example describes four experiments in which a compound of formula I, was administered in combination with either a typical (exemplified by haloperidol) or atypical (exemplified by clozapine). The combination reduced apomorphine-induced climbing without side effects.

Experiment 1

| Animal | | Number of |
|---|---|---|
| groups | Treatment | animals (n) |
| 1 | vehicle + vehicle | 6 |
| 2 | vehicle + cpd 1 (1.7 mg/kg) | 6 |
| 3 | vehicle + cpd 1 (5.4 mg/kg) | 6 |
| 4 | vehicle + cpd 1 (17 mg/kg) | 6 |
| 5 | Haloperidol (0.17 mg/kg) + vehicle | 6 |
| 6 | Haloperidol (0.17 mg/kg) + cpd 1 (1.7 mg/kg) | 6 |
| 7 | Haloperidol (0.17 mg/kg) + cpd 1 (5.4 mg/kg) | 6 |
| 8 | Haloperidol (0.17 mg/kg) + cpd 1 (17 mg/kg) | 6 |
| 9 | Haloperidol (0.3 mg/kg) + vehicle | 6 |
| 10 | Haloperidol (0.3 mg/kg) + cpd 1 (1.7 mg/kg) | 6 |
| 11 | Haloperidol (0.3 mg/kg) + cpd 1 (5.4 mg/kg) | 6 |
| 12 | Haloperidol (0.3 mg/kg) + cpd 1 (17 mg/kg) | 6 |

Experiment 2

| Animal | | Number |
|---|---|---|
| groups | Treatment | of animals (n) |
| 1 | vehicle + vehicle | 6 |
| 2 | vehicle + cpd 1 (1.7 mg/kg) | 6 |
| 3 | vehicle + cpd 1 (5.4 mg/kg) | 6 |
| 4 | vehicle + cpd 1 (17 mg/kg) | 6 |
| 5 | Clozapine (3 mg/kg) + vehicle | 6 |
| 6 | Clozapine (3 mg/kg) + cpd 1 (1.7 mg/kg) | 6 |
| 7 | Clozapine (3 mg/kg) + cpd 1 (5.4 mg/kg) | 6 |
| 8 | Clozapine (3 mg/kg) + cpd 1 (17 mg/kg) | 6 |
| 9 | Clozapine (5.4 mg/kg) + vehicle | 6 |
| 10 | Clozapine (5.4 mg/kg) + cpd 1 (1.7 mg/kg) | 6 |
| 11 | Clozapine (5.4 mg/kg) + cpd 1 (5.4 mg/kg) | 6 |
| 12 | Clozapine (5.4 mg/kg) + cpd 1 (17 mg/kg) | 6 |

Experiment 3

| Animal | | Number |
|---|---|---|
| groups | Treatment | of animals (n) |
| 1 | vehicle + vehicle | 6 |
| 2 | vehicle + Cpd 2 (3 mg/kg) | 6 |
| 3 | vehicle + Cpd 2 (10 mg/kg) | 6 |
| 4 | vehicle + Cpd 2 (30 mg/kg) | 6 |
| 5 | Haloperidol (0.1 mg/kg) + vehicle | 6 |
| 6 | Haloperidol (0.1 mg/kg) + Cpd 2 (3 mg/kg) | 6 |
| 7 | Haloperidol (0.1 mg/kg) + Cpd 2 (10 mg/kg) | 6 |
| 8 | Haloperidol (0.17 mg/kg) + Cpd 2 (30 mg/kg) | 6 |
| 9 | Haloperidol (0.17 mg/kg) + vehicle | 6 |
| 10 | Haloperidol (0.17 mg/kg) + Cpd 2 (3 mg/kg) | 6 |
| 11 | Haloperidol (0.17 mg/kg) + Cpd 2 (10 mg/kg) | 6 |
| 12 | Haloperidol (0.17 mg/kg) + Cpd 2 (30 mg/kg) | 6 |

Experiment 4

| Animal | | Number |
|---|---|---|
| groups | Treatment | of animals (n) |
| 1 | vehicle + vehicle | 6 |
| 2 | vehicle + Cpd 2 (3 mg/kg) | 6 |
| 3 | vehicle + Cpd 2 (10 mg/kg) | 6 |
| 4 | vehicle + Cpd 2 (30 mg/kg) | 6 |
| 5 | Clozapine (3 mg/kg) + vehicle | 6 |
| 6 | Clozapine (3 mg/kg) + Cpd 2 (3 mg/kg) | 6 |
| 7 | Clozapine (3 mg/kg) + Cpd 2 (10 mg/kg) | 6 |
| 8 | Clozapine (3 mg/kg) + Cpd 2 (30 mg/kg) | 6 |
| 9 | Clozapine (5.4 mg/kg) + vehicle | 6 |
| 10 | Clozapine (5.4 mg/kg) + Cpd 2 (3 mg/kg) | 6 |
| 11 | Clozapine (5.4 mg/kg) + Cpd 236 (10 mg/kg) | 6 |
| 12 | Clozapine (5.4 mg/kg) + Cpd 2 (30 mg/kg) | 6 |

Procedure:

Mice were acclimated to the climbing cages for at least 1 hour and then dosed with either the vehicle or a dose of haloperidol or clozapine followed by a dose of either vehicle or a dose of Compound 1. Thirty minutes after dosing all mice received 1 mg/kg, s.c. apomorphine and returned to the climbing cages. Five minutes after apomorphine the mice were observed and scored for climbing and stereotypy every 5 minutes for the 30 minute test session.

Results

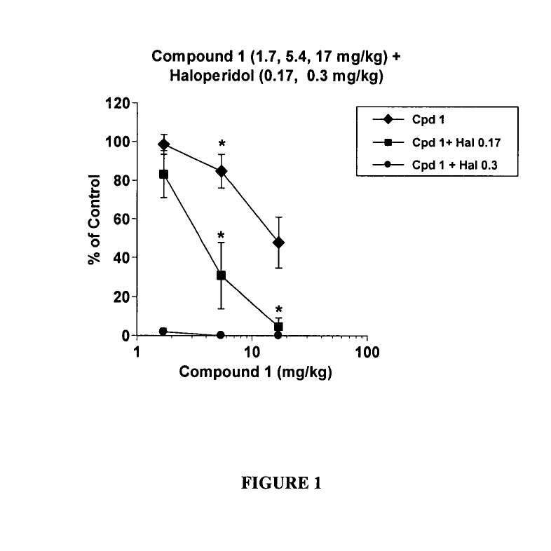

Experiment 1: Compound 1 (5.4 & 17 mg/kg) when co-administered with haloperidol (0.17 & 0.3 mg/kg) produced a greater block of apomorphine-induced climbing then when either compound was administered alone with modest decreases (<10%) in stereotypy in the 17 mg/kg Compound 1 and 0.17 mg/kg haloperidol combined treatment group. With the 0.3 mg/kg haloperidol dose, all doses of Compound 1 resulted in decreased stereotypy relative to 0.3 haloperidol alone. The degree of potentiated side-effect liability was tested separately in the catalepsy assay (see section II below). The ED₅₀ for apomorphine block for compound 1 alone was 15.89 mg/kg and was reduced to 4.53 mg/kg when co-administered with 0.17 mg/kg of haloperidol, while an ED₅₀ with 0.3 mg/kg of haloperidol could not be calculated (FIG. 1).

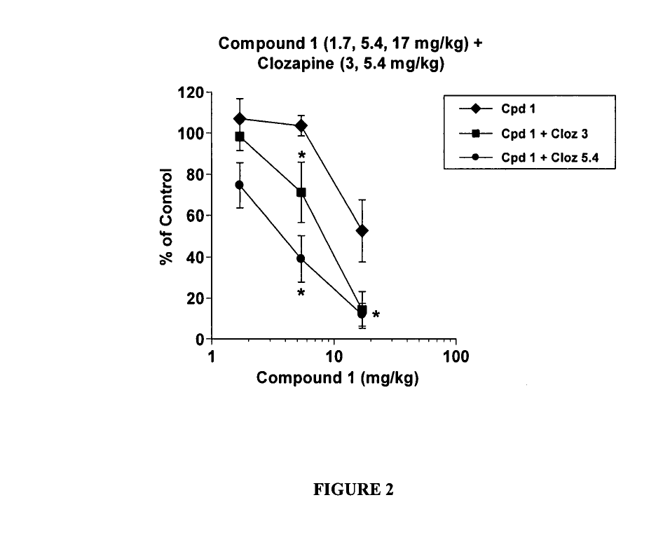

Experiment 2: Compound 1 (5.4 & 17 mg/kg) when co-administered with clozapine (3 & 5.4 mg/kg) produced a greater block of apomorphine-induced climbing then when either compound was administered alone with only a slight decrease (˜13%) in stereotypy in the 17 mg/kg Compound 1 and 5.4 mg/kg clozapine combined treatment. The ED₅₀ for apomorphine block for Compound 1 alone was 17.03 mg/kg and was reduced to 8.19 and 3.96 mg/kg when co-administered with 3 and 5.4 mg/kg of Clozapine respectively (FIG. 2).

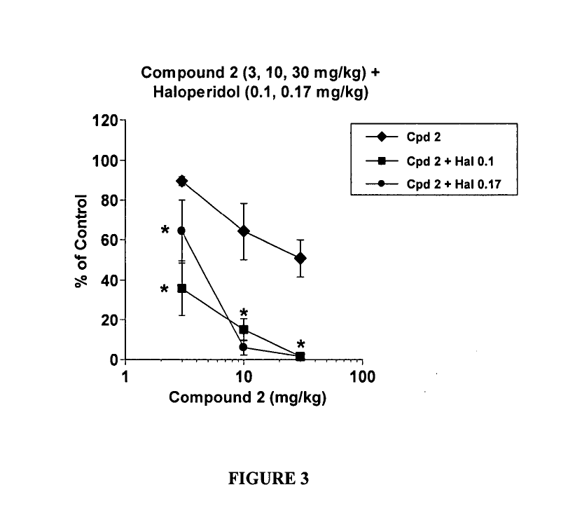

Experiment 3: Compound 2 (3, 10 & 30 mg/kg) when co-administered with haloperidol (0.1 & 0.17 mg/kg) produced a greater block of apomorphine-induced climbing then when either compound was administered alone with only a slight decrease in stereotypy (˜30%) at the 30 mg/kg Compound 2 and 0.17 mg/kg haloperidol combined treatment relative to haloperidol alone. The ED₅₀ for apomorphine block for Compound 2 alone was 27.06 mg/kg and was reduced to 2.4 and 4.1 mg/kg when co-administered with 0.1 and 0.17 mg/kg of haloperidol respectively (FIG. 3).

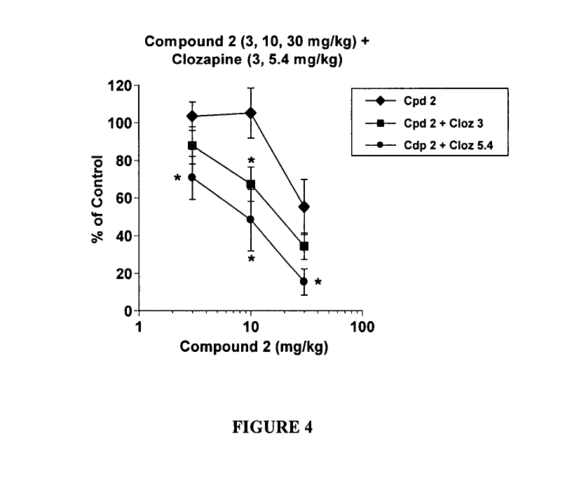

Experiment 4: Compound 2 (3, 10 & 30 mg/kg) when co-administered with clozapine (3 & 5.4 mg/kg) produced a greater block of apomorphine-induced climbing then when either compound was administered alone with only a slight decrease in stereotypy (˜20%) at the 30 mg/kg Compound 2 and 5.4 mg/kg clozapine combined treatment. The ED₅₀ for apomorphine block for Compound 2 alone was 27.06 and was reduced to 17.9 and 8.33 when co-administered with 3 and 5.4 mg/kg of clozapine respectively (FIG. 4).

### 2. Example 2

### Extrapyramidal Side Effects of Inventive Combinations As Compared with Anti-Psychotic Agents Alone

Using 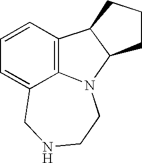
(Compound 1, “cpd 1”) and

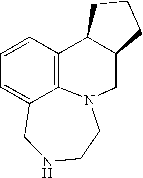

(Compound 2, “cpd 2”) to exemplify, the present Example describes an experiment in which a compound of formula I was administered in combination with haloperidol to assess extra-pyramidal side effect liability, as represented by cataleptic behavior.

Experiment 1

| Animal | | Number |
|---|---|---|
| groups | Treatment | of animals (n) |
| 1 | vehicle + vehicle | 6 |
| 2 | vehicle + cpd 1 (5.4 mg/kg) | 6 |
| 3 | vehicle + cpd 1 (17 mg/kg) | 6 |
| 4 | Haloperidol (0.17 mg/kg) + vehicle | 6 |
| 5 | Haloperidol (0.17 mg/kg) + cpd 1 (1.7 mg/kg) | 6 |
| 6 | Haloperidol (0.17 mg/kg) + cpd 1 (5.4 mg/kg) | 6 |
| 7 | Haloperidol (0.17 mg/kg) + cpd 1 (17 mg/kg) | 6 |

Experiment 2

| Animal | | Number |
|---|---|---|
| groups | Treatment | of animals (n) |
| 1 | vehicle + vehicle | 6 |
| 2 | vehicle + cpd 1 (1.7 mg/kg) | 6 |
| 3 | vehicle + cpd 1 (5.4 mg/kg) | 6 |
| 4 | vehicle + cpd 1 (17 mg/kg) | 6 |
| 5 | Haloperidol (0.3 mg/kg) + vehicle | 6 |
| 6 | Haloperidol (0.3 mg/kg) + cpd 1 (1.7 mg/kg) | 6 |
| 7 | Haloperidol (0.3 mg/kg) + cpd 1 (5.4 mg/kg) | 6 |
| 8 | Haloperidol (0.3 mg/kg) + cpd 1 (17 mg/kg) | 6 |

Experiment 3

| Animal | | Number |
|---|---|---|
| groups | Treatment | of animals (n) |
| 1 | vehicle + vehicle | 6 |
| 2 | vehicle + cpd 2 (10 mg/kg) | 6 |
| 3 | Haloperidol (0.1 mg/kg) + vehicle | 6 |
| 4 | Haloperidol (0.17 mg/kg) + vehicle | 6 |
| 5 | Haloperidol (0.1 mg/kg) + cpd 2 (10 mg/kg) | 6 |
| 6 | Haloperidol (0.17 mg/kg) + cpd 2 (10 mg/kg) | 6 |

Procedure:

Mice were dosed with either the vehicle or a dose of haloperidol followed by a dose of either vehicle or a dose of Compound 1 or Compound 2. Thirty, 60, 90 and 120 minutes after dosing, the forelegs are draped over a thin horizontal rod 1.75″ high. The number of seconds up to 60 sec for which the mouse remains on the bar is recorded at each test point and presented as the percent of maximum possible response (60 sec).

Results:

Experiments 1 and 2: Compound 1 (17 mg/kg) resulted in a moderate increase in catalepsy induced by 0.17 and 0.3 mg/kg haloperidol up to 30% of maximum catalepsy while the lower doses of Compound 1 failed to potentiate catalepsy. Thus, there is a large window for the increased efficacy (5.4 mg/kg) with co-administration and increased EPS liability (≧17 mg/kg).

Experiment 3: Compound 2 (10 mg/kg) failed to potentiate haloperidol (0.1 and 0.17 mg/kg) induced catalepsy. Thus, there is a large window for the increased efficacy (3 mg/kg) with co-administration and increased EPS liability (>10 mg/kg).

### 3. Example 3

### Inventive Combinations on Conditioned Avoidance Responding As Compared with Anti-Psychotic Agents Alone

Using 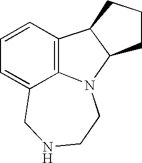
(Compound 1, “cpd 1”) and

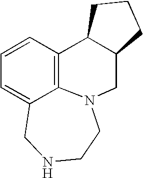

(Compound 2, “cpd 2”) to exemplify, the present Example describes an experiment to determine the effect of co-administration of haloperidol (0.54 mg/kg, i.p. 30′ pretreatment) or clozapine (5.4 mg/kg, i.p., 30′ pretreatment) and compound 1 (0.54 mg/kg, i.p., 30′ pretreatment) or compound 2 (1 mg/kg, i.p. 30′ pretreatment) on conditioned avoidance responding.

Experiment 1

| Animal | | Number |
|---|---|---|
| groups | Treatment | of animals (n) |
| 1 | vehicle + vehicle | 8 |
| 2 | vehicle + cpd 1 (0.54 mg/kg) | 8 |
| 3 | Haloperidol (0.54 mg/kg) + vehicle | 8 |
| 4 | Haloperidol (0.54 mg/kg) + cpd 1 (0.54 mg/kg) | 8 |

Experiment 2

| Animal | | Number |
|---|---|---|
| groups | Treatment | of animals (n) |
| 1 | vehicle + vehicle | 8 |
| 2 | vehicle + cpd 1 (0.54 mg/kg) | 8 |
| 3 | Clozapine (5.4 mg/kg) + vehicle | 8 |
| 4 | Clozapine (5.4 mg/kg) + cpd 1 (0.54 mg/kg) | 8 |

Experiment 3

| Animal | | Number |
|---|---|---|
| groups | Treatment | of animals (n) |
| 1 | vehicle + vehicle | 8 |
| 2 | vehicle + cpd 2 (1 mg/kg) | 8 |
| 3 | Haloperidol (0.54 mg/kg) + vehicle | 8 |
| 4 | Haloperidol (0.54 mg/kg) + cpd 2 (1 mg/kg) | 8 |

Experiment 4

| | | Number of |
|---|---|---|
| Animal groups | Treatment | animals (n) |
| 1 | vehicle + vehicle | 8 |
| 2 | vehicle + cpd 2 (1 mg/kg) | 8 |
| 3 | Clozapine (5.4 mg/kg) + vehicle | 8 |
| 4 | Clozapine (5.4 mg/kg) + cpd 2 (1 mg/kg) | 8 |

Procedure:

Male rats had been trained to a stable criterion performance of 90% avoidance responding and had been used in previous drug studies. The dosing plan was a within subject pseudo random design so that all rats received all treatments over several weeks. On test days in experiments 1 and 2 the rats were dosed with vehicle, Compound 1, haloperidol or clozapine or a combination of Compound 1/haloperidol or Compound 1/clozapine. On test days in experiments 3 and 4 the rats were dosed with vehicle, Compound 2, haloperidol or clozapine or a combination of Compound 2/haloperidol or Compound 2/clozapine. Conditioned avoidance testing commenced 30 minutes after treatment and the rats received 50 trials and the number of avoidance, escape and no response trials was recorded.

Results

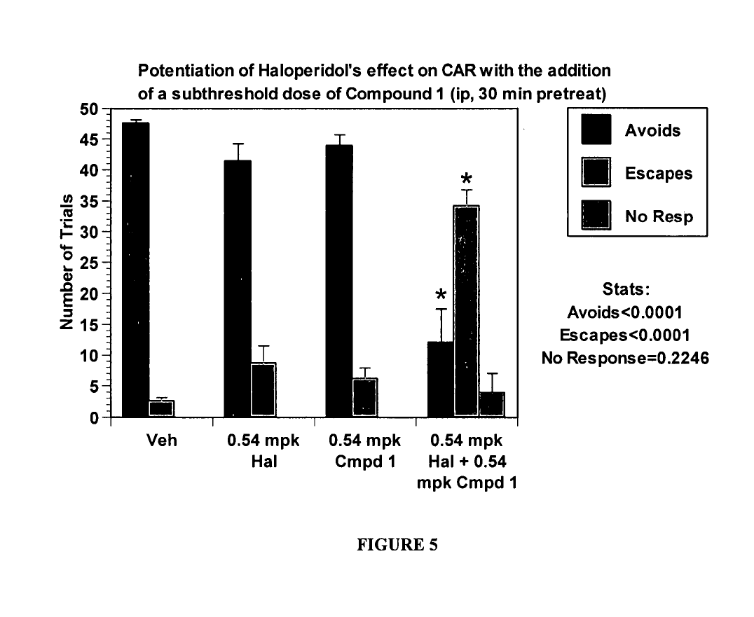

Experiment 1: Individually, Compound 1 (0.54 mg/kg) and haloperidol (0.54 mg/kg) resulted in a slight decrease in avoidance responding (˜10%). When co-administered at these minimally effective doses, Compound 1 and haloperidol resulted in a >70% decrease in avoidance responding with no adverse effects on number of no response trials (FIG. 5).

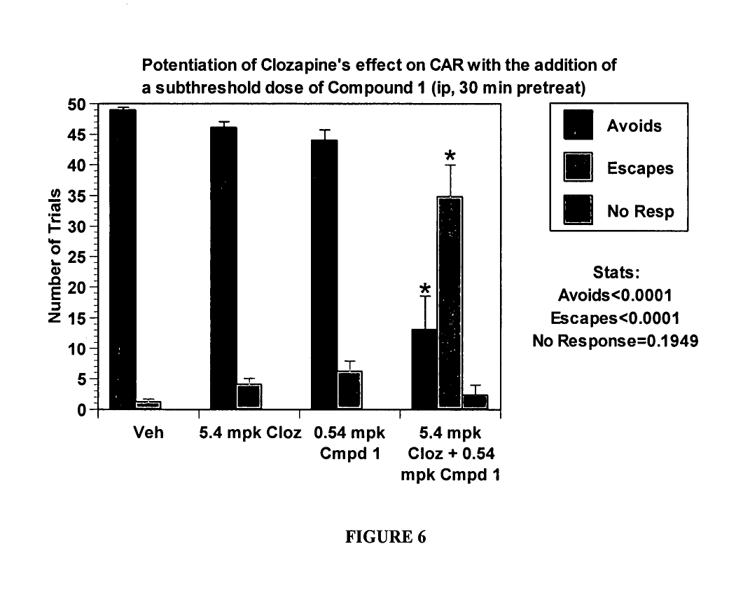

Experiment 2: Individually, Compound 1 (0.54 mg/kg) and clozapine (5.4 mg/kg) resulted in a slight decrease in avoidance responding (˜10%). When co-administered at these minimally effective doses, Compound 1 and clozapine resulted in a >70% decrease in avoidance responding with no adverse effects on number of no response trials (FIG. 6).

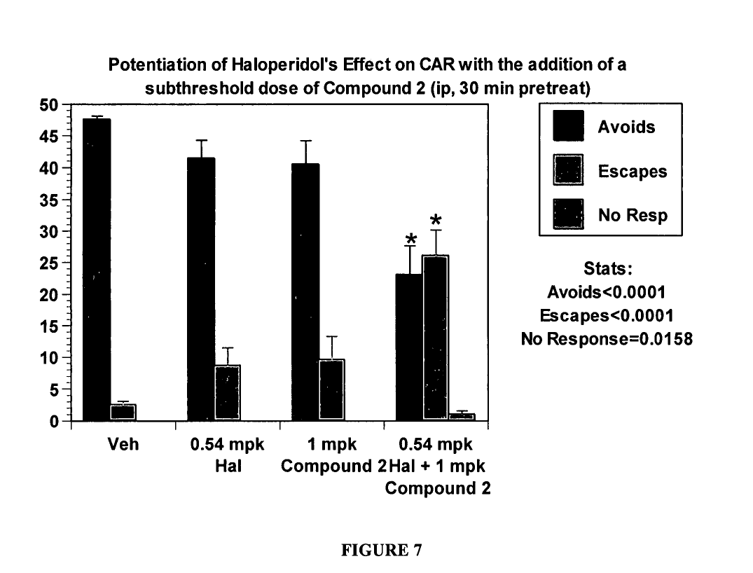

Experiment 3: Individually, Compound 2 (1 mg/kg) and haloperidol (0.54 mg/kg) resulted in a slight decrease in avoidance responding (˜10-20%). When co-administered at these minimally effective doses, Compound 2 and haloperidol resulted in a >50% decrease in avoidance responding with no adverse effects on number of no response trials (FIG. 7).

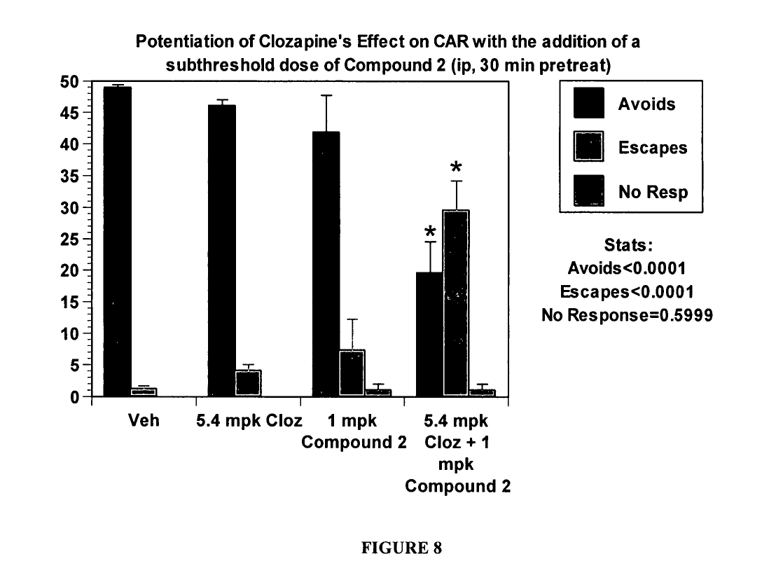

Experiment 4: Individually, Compound 2 (1 mg/kg) and clozapine (5.4 mg/kg) resulted in a slight decrease in avoidance responding (˜10-20%). When co-administered at these minimally effective doses, Compound 2 and clozapine resulted in a >60% decrease in avoidance responding with no adverse effects on number of no response trials (FIG. 8).

Thus, the present Examples illustrate that inventive combinations enhance the ability of haloperidol to treat positive symptoms of schizophrenia as modeled by the amphetamine-induced hyperactivity with acceptable liability for the side effects it induces as modeled by catelepsy.

The entire disclosure of each patent, patent application, and publication cited or described in this document is hereby incorporated by reference.

While we have presented a number of embodiments of this invention, it is apparent that our basic construction can be altered to provide other embodiments which utilize the compounds and methods of this invention. Therefore, it will be appreciated that the scope of this invention is to be defined by the appended claims rather than by the specific embodiments which have been represented by way of example.

What is claimed is:

1. A composition comprising:
(a) either a typical or atypical anti-psychotic drug;
(b) a compound of formula I: 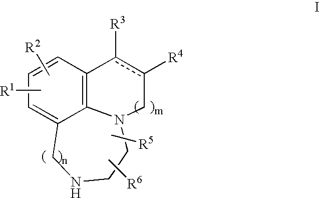
or a pharmaceutically acceptable salt thereof, wherein: [symbol]
designates a single or double bond;
n is 1 or2;
m is 0 or 1;
R¹ and R² are each independently halogen, —CN, —R, —OR, —C₁₋₆ perfluoroalkyl, or —OC_{1- 6} perfluoroalkyl;
each R is independently hydrogen or a C₁₋₆ alkyl group;
R³ and R⁴ are taken together, with the carbon atoms to which they are bound, to form a saturated or unsaturated 4-8 membered ring, wherein said ring is optionally substituted with 1-3 groups independently selected from halogen, —R, or OR; and
R⁵ and R⁶ are each independently —R; and
(c) optionally a pharmaceutically acceptable carrier, adjuvant, or vehicle.

2. The composition according to claim 1, wherein [symbol] designates a single bond.

3. The composition according to claim 1, wherein:
R¹ is R, OR, halogen, cyano, or —C₁₋₃ perfluoroalkyl; and
R² is R, OR, halogen, cyano, or —C₁₋₃ perfluoroalkyl.

4. The composition according to claim 3, wherein at least one of R¹ and R² is —OH.

5. The composition according to claim 3, wherein R³ and R⁴ are taken together, with the carbon atoms to which they are bound, to form a saturated or unsaturated 5-8 membered ring, wherein said ring is optionally substituted with 1-3 groups independently selected from halogen, —R, or OR.

6. The composition according to claim 1, wherein said compound is of formula I-a or I-b: 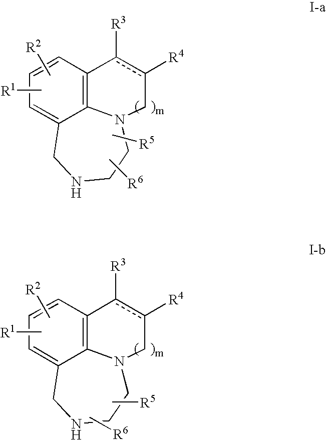
or a pharmaceutically acceptable salt thereof.

7. The composition according to claim 1, wherein said compound is of formula I-c or I-d: 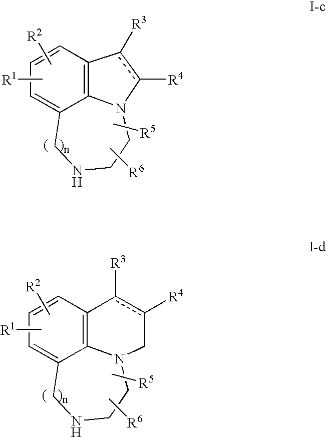
or a pharmaceutically acceptable salt thereof.

8. The composition according to claim 7, wherein said compound is of formula II or III: 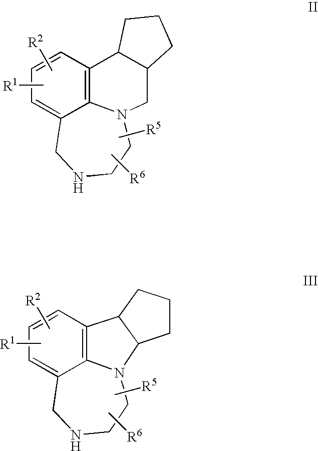
or a pharmaceutically acceptable salt thereof.

9. The composition according to claim 1, wherein said compound is of formula I-e or I-f: 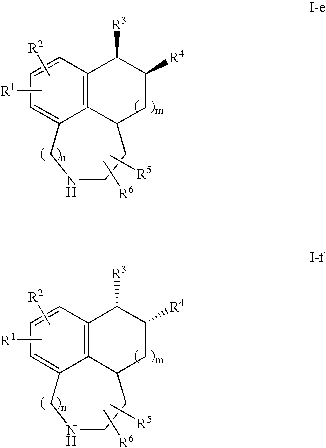
or a pharmaceutically acceptable salt thereof.

10. The composition according to claim 9, wherein said compound is of formula IV or V: 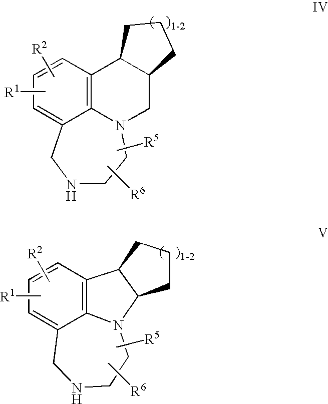
or a pharmaceutically acceptable salt thereof.

11. The composition according to claim 1, wherein said compound is selected from:
2-bromo-4,5,6,7,9,9a,10,11,12,12a-decahydrocyclopenta[c][1,4]diazepino [6,7,1-ij]quinoline;
2-bromo-4,5,6,7,9,9a,10,11,12,13,14,14a-dodecahydrocyclohepta[c][1,4 ]diazepino[6,7,1-ij]quinoline;
2-chloro-4,5,6,7,9,9a,10,11,12,12a-decahydrocyclopenta[c][1,4]diazepino [6,7,1-ij]quinoline;
2-chloro-4,5,6,7,9,9a,10,11,12,13,14,14a-dodecahydrocyclohepta[c][1,4] diazepino[6,7,1-ij]quinoline;
2-phenyl-4,5,6,7,9,9a,10,11,12,12a-decahydrocyclopenta[c][1,4]diazepino [6,7,1-ij]quinoline;
2-methoxy-4,5,6,7,9,9a,10,11,12,12a-decahydrocyclopenta[c][1,4]diazepino [6,7,1-ij]quinoline;
1-fluoro-4,5,6,7,9,9a,10,11,12,12a-decahydrocyclopenta[c][1,4] diazepino [6,7,1-ij] quinoline;
1-fluoro-4,5,6,7,9,9a,10,11,12,13,14,14a-dodecahydrocyclohepta[c][1,4] diazepino[6,7,1-ij]quinoline;
1-(trifluoromethyl)-4,5,6,7,9,9a,10,11,12,12a-decahydrocyclopenta[c][1,4] diazepino [6,7,1-ij]quinoline;
1-fluoro-2-methoxy-4,5,6,7,9,9a,10,11,12,12a-decahydrocyclopenta[c][1,4] diazepino[6,7,1-ij]quinoline;
1-fluoro-2-methoxy-4,5,6,7,9,9a,10,11,12,13,14,14a-dodecahydrocyclo-hepta[c][1,4]diazepino[6,7,1-ij]quinoline;
4,5,6,7,9,9a 10,11,12,12a-decahydrocyclopenta[c][1,4]diazepino[6,7,1-ij] quinoline;
4,5,6,7,9,9a,10,11,12,13,14,14a-dodecahydrocyclohepta[c][1,4]diazepino [6,7,1-ij]quinoline;
(−)-4,5,6,7,9,9a 10,11,12,12a-decahydrocyclopenta[c][1,4]diazepino [6,7,1-ij] quinoline;
(9aR, 14aS)-4,5,6,7,9,9a,10,11,12,13,14,14a-dodecahydrocyclohepta[c][1,4] diazepino[6,7,1-ij]quinoline;
(9aS, 14aR)-4,5,6,7,9,9a,10,11,12,13,14,14a-dodecahydrocyclohepta[c][1,4] diazepino[6,7,1-ij]quinoline;
4,5,6,7,9a,10,11,12,13,13a-decahydro-9H-[1,4]diazepino[6,7,1-de]phenanthridine;
1,2,3,4,9,10-hexahydro-8H-cyclopenta[b][1,4]diazepino[6,7,-hi]indole;
1,2,3,4,8,9,10,10a-octahydro-7bH-cyclopenta[b][1,4]diazepino[6,7,1-hi]indole;
(7bS,10aS)-1,2,3,4,8,9,10,10a-octahydro-7bH-cyclopenta[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
(7bR,10aR)-1,2,3,4,8,9,10,10a-octahydro-7bH-cyclopenta-[b][1,4]diazepino[6,7,1-hi]indole;
6-methyl-1,2,3,4,9,10-hexahydro-8H-cyclopenta[b][1,4]diazepino[6,7,1-hi]indole;
2S)-(rel-7bR, 10aR)-2-methyl-1,2,3,4,8,9,10,10a-octahydro-7bH-cyclopenta[b][1,4]diazepino[6,7,1-hi]indole;
(2S)-(rel-7bR,10aR)-2-methyl-1,2,3,4,8,9,10,10a-octahydro-7bH-cyclopenta[b][1,4]diazepino[6,7,1-hi]indole;
(2S)-(rel-7bS,10aS)-2-methyl-1,2,3,4,8,9,10,10a-octahydro-7bH-cyclopenta[b][1,4]diazepino[6,7,1-hi]indole;
(2R)-(rel-7bR, 10aR)-2-methyl-1,2,3,4,8,9,10,10a-octahydro-7bH-cyclopenta[b][1,4]diazepino[6,7,1-hi]indole;
(2R)-(rel-7bR,10aR)-2-methyl-1,2,3,4,8,9,10,10a-octahydro-7bH-cyclopenta[b ][1,4]diazepino[6,7,1-hi]indole;
(2R)-(rel-7bS,10aS)-2-methyl-1,2,3,4,8,9,10,10a-octahydro-7bH-cyclopenta[b][1,4]diazepino[6,7,1-hi]indole;
rel-(4S,7bS,10aS)-4-methyl-1,2,3,4,8,9,10,10a-octahydro-7bH-cyclopenta[b][1,4] diazepino[6,7,1-hi]indole
rel-(4S ,7bS,10aS)-4-methyl-1,2,3,4,8,9,10,10a-octahydro-7bH-cyclopenta[b]-[1,4]diazepino[6,7,1-hi]indole;
rel-(4R,7bS,10aS)-4-methyl-1,2,3,4,8,9,10,10a-octahydro-7bH-cyclopenta[b][1,4]diazepino[6,7,1-hi]indole;
9-methyl-1,2,3,4,8,9,10,10a-octahydro-7bH-cyclopenta[b][1,4]diazepino[6,7,1-hi]indole;
(7bR,9R,10aR)-9-methyl-1,2,3,4,8,9,10,10a-octahydro-7bH-cyclopenta[b][1,4]d iazepino[6,7,1-hi]indole;
9,9-dimethyl-1,2,3,4,8,9,10,10a-octahydro-7bH-cyclopenta[1,4]diazepino[6,7,1-hi]indole;
(7bR,10aR)-9,9-dimethyl-1,2,3,4,8,9,10,10a-octahydro-7bH-cyclopenta[b][1,4] diazepino[6,7,1-hi]indole; and
(7bS,10aS)-9,9-dimethyl-1,2,3,4,8,9,10,10a-octahydro-7bH-cyclopenta[b][1,4] diazepino[6,7,1-hi]indole;
or a pharmaceutically acceptable salt thereof.

12. The composition of claim 11, wherein said compound is the hydrochloride salt.

13. A method of treating a patient suffering from a psychotic disorder comprising administering to said patient the composition according to claim 1.

14. The method according to claim 13, wherein the anti-psychotic agent is an atypical anti-psychotic.

15. The method according to claim 13, wherein the anti-psychotic agent is a typical anti-psychotic.

16. The method according to claim 13, wherein the anti-psychotic agent is selected from chlorpromazine, mesoridazine, thioridazine, fluphenazine, trifluoperazine, perphenazine, clozapine, haloperidol, loxapine, molindone, thiothixene, risperidone, seroquel, or olanzapine.

17. The method according to claim 13, wherein administration of the composition is oral.

18. The method according to claim 13, wherein the patient is suffering from schizophrenia.

19. The method according to claim 13, wherein the patient is suffering from schizoaffective disorder.

20. The method according to claim 13, wherein the patient is suffering from bipolar disorder.
